(19) United States
(12) Patent Application Publication
(10) Pub. No.: US 2024/0066207 A1
(43) Pub. Date: Feb. 29, 2024

(54) SYSTEMS AND METHODS FOR CONTROLLING PARTIAL DELIVERY PUMPING IN INFUSION SYSTEMS

(71) Applicants: Baxter International Inc., Deerfield, IL (US); Baxter Healthcare SA, Glattpark (Opfikon) (CH)

(72) Inventors: Saman Khan, Mumbai (IN); Rajesh Swarnkar, Bangalore (IN); Prasad Digaskar, Mumbai (IN)

(21) Appl. No.: 18/237,235

(22) Filed: Aug. 23, 2023

(30) Foreign Application Priority Data
Aug. 26, 2022 (IN) 202241048929

(51) Int. Cl. A61M 5/142

(52) U.S. Cl. CPC A61M 5/142; A61M 2005/14208; A61M 2205/50

(57) ABSTRACT

A method and system for delivering partial boluses using infusion pumps is disclosed. An example method includes storing a programmed dose volume to be administered in response to a request from a patient and instructing the pump to begin dispensing the programmed dose volume of fluid from the fluid supply. The method further includes receiving an indication that the pump has stopped pumping, determining a partial dose volume, and when the partial dose volume equals a value greater than zero, generate an alarm that the fluid supply is depleted. Additionally, the method includes exchanging the depleted fluid supply with a subsequent fluid supply, dispensing from the subsequent fluid supply the partial dose volume, and preventing any subsequent dispensing of fluid from the subsequent fluid supply for a programed lockout period.

### PRIORITY CLAIM

This application claims priority to Indian Provisional Patent Application No. 202241048929 filed Aug. 26, 2022, entitled “SYSTEMS AND METHODS FOR CONTROLLING PARTIAL DELIVERY PUMPING IN INFUSION SYSTEMS,” which is which is incorporated herein by reference in its entirety.

### TECHNICAL FIELD

The instant application is directed towards electronic devices and more specifically to control systems for controlling the performance of infusion pump systems.

### BACKGROUND

The present disclosure provides new and innovative methods and systems for fluid delivery control in electronic devices, including medical devices. In various embodiments, the device includes an infusion pump. Generally, medical patients sometimes require precise delivery of either medication at set periodic intervals or medication delivery on demand via a patient initiated request. Medical pumps have been developed to provide controlled drug infusion wherein the drug can be administered at a precise dose that keeps the drug concentration within a therapeutic margin and out of an unnecessary or possibly toxic range. The medical pumps can provide appropriate drug delivery to the patient at a controllable dose, which does not require frequent attention.

Configurations of infusion pumps include elastomeric pumps, which squeeze solution from flexible containers, such as balloons, into IV tubing for delivery to the patient. Alternatively, spring-loaded pumps pressurize the solution containers or reservoirs. Certain pump designs utilize cartridges containing flexible compartments that are squeezed by pressure rollers for discharging the solutions. Infusion pumps utilizing syringes are also known wherein a drive mechanism moves a plunger of the syringe to deliver fluid to a patient. Typically, these infusion pumps include a housing adapted to receive a syringe assembly, a drive mechanism adapted to move the syringe plunger, a pump control unit having a variety of operating controls, and a power source for powering the pump including the drive mechanism and controls.

Medical pumps may facilitate administration of intravenous therapy to patients both in and outside of a clinical setting. One type of medical pumps inside the clinical setting is a Patient-Controlled Analgesia (PCA) pump. PCA pumps typically deliver pain medication and allow patients to request medication delivery from the PCA pump, referred to as a PCA bolus, via a bolus cord attached to the pump. The PCA pump may also deliver pre-programmed intermittent boluses by programming the PCA pump to deliver a specific bolus volume with a specific time interval between two boluses. However, when a patient requests a PCA bolus and the volume remaining in the syringe is less than the requested bolus volume, the pump does not deliver any drug since the requested volume is not available. The patient cannot receive the entire programmed bolus volume requested and a clinician may or may not be present to address the patient in pain. This results in unannounced interruption in therapy or insufficient therapy resulting in significant decrease of pharmacological effects and worsening of patient condition.

Another disadvantage of existing PCA pump control systems is the wastage of remaining volume of medication, typically an opioid, in the syringe. Hospitals follow strict protocols to track the volume of opioids administered or discarded to prevent unauthorized access to opioids. In this case, since the clinician will have to discard the remaining volume of drug, there is a possibility of the drug misuse and in the absence of accountability measures or systems. Additionally, inefficiencies and the wastefulness of discarding the residual volume of drug become apparent when scaled to account for multiple patients across a hospital system. This wastefulness contributes to the depilation of already scarce resources in a hospital system.

Several methods exist to ensure that a medical device delivers medication at a specified rate. However, the existing methods have several disadvantages, limitations, and drawbacks. For example, existing methods utilize alarms to alert a clinician that the volume remaining in the syringe is less than the requested bolus volume and, accordingly, the syringe needs to be replaced with a full syringe. However, this results in an unannounced interruption in therapy or insufficient therapy resulting in significant decrease of pharmacological effects because of the time required for the syringe to replaced and the clinician to enter instructions into the PCA pump. This can be particularly problematic for instances where every second without medication counts, for example, when the patient is in severe pain immediately after surgery. Accordingly, to overcome these problems and drawback, a method and system for controlling the operation of pumps and managing the delivery of partial medication doses to eliminate the downtime and inefficiencies associated with partial medication delivery is desired.

### SUMMARY

The present disclosure provides new and innovative methods and systems for controlling the operation of pumps and managing the delivery of partial medication doses such as PCA pumps. In various embodiments, a computer-implemented method includes storing, in a memory of a pump, a programmed dose volume of a fluid from a fluid supply to be administered in response to a request from an operator; and when the request to dispense the programmed dose volume is received in a controller of the pump, instructing, via the controller, the pump to begin dispensing the programmed dose volume of the fluid from the fluid supply.

In another aspect of the present disclosure, which may be used in combination with any other aspect or combination of aspects listed herein the computer-implemented method includes instructing, via the controller, the pump to receive an indication that the pump has stopped pumping.

In another aspect of the present disclosure, which may be used in combination with any other aspect or combination of aspects listed herein the computer-implemented method includes instructing, via the controller, the pump to determine a partial dose volume equal to a difference in volume between the programmed dose and a volume of the fluid actually dispensed from the fluid supply.

In another aspect of the present disclosure, which may be used in combination with any other aspect or combination of aspects listed herein the computer-implemented method includes instructing, via the controller, the pump to when the partial dose volume equals zero, repeat in response to a subsequent request from the patient when a programmed lockout period has elapsed.

In another aspect of the present disclosure, which may be used in combination with any other aspect or combination of aspects listed herein the computer-implemented method includes instructing, via the controller, the pump to when the partial dose volume equals a value greater than zero, generate an alarm that the fluid supply is depleted and record, in a history log stored in the memory, the difference in volume between the programmed dose and the volume of the fluid dispensed from the fluid supply.

In another aspect of the present disclosure, which may be used in combination with any other aspect or combination of aspects listed herein the computer-implemented method includes instructing, via the controller, the pump to, in response to the alarm that the fluid supply is depleted, provide a prompt to exchange the depleted fluid supply with a subsequent fluid supply.

In another aspect of the present disclosure, which may be used in combination with any other aspect or combination of aspects listed herein the computer-implemented method includes instructing, via the controller, the pump to after receiving an indication that the subsequent fluid supply is fluidly coupled to the pump, dispense, from the subsequent fluid supply, the partial dose volume.

In another aspect of the present disclosure, which may be used in combination with any other aspect or combination of aspects listed herein the computer-implemented method includes instructing, via the controller, the pump to prevent any subsequent dispensing of fluid from the subsequent fluid supply for the programed lock out duration.

In another aspect of the present disclosure, which may be used in combination with any other aspect or combination of aspects listed herein the computer-implemented method includes wherein the fluid supply and the subsequent fluid supply comprise the same fluid.

In another aspect of the present disclosure, which may be used in combination with any other aspect or combination of aspects listed herein the computer-implemented method includes wherein the fluid supply and the subsequent fluid supply are housed in a syringe configured to engage with the pump.

In another aspect of the present disclosure, which may be used in combination with any other aspect or combination of aspects listed herein the computer-implemented method includes wherein the fluid supply and the subsequent fluid supply contain an analgesic.

In another aspect of the present disclosure, which may be used in combination with any other aspect or combination of aspects listed herein the computer-implemented method includes wherein the controller of the pump, instruct the pump to repeat the method in response to a subsequent request from the patient if the programmed lockout period has elapsed.

In various embodiments, an infusion pump apparatus includes a pumping mechanism configured to dispense a programmed dose volume from a fluid supply, a memory storing the programmed dose volume and configured to record a history log, a patient request device.

In another aspect of the present disclosure, which may be used in combination with any other aspect or combination of aspects listed herein the infusion pump apparatus includes a processor in communication with the pumping mechanism, the memory and the patient request device.

In another aspect of the present disclosure, which may be used in combination with any other aspect or combination of aspects listed herein the infusion pump apparatus includes the processor configured to, when the patient has made a request, via the patient request device, dispense the programmed dose volume from the fluid supply.

In another aspect of the present disclosure, which may be used in combination with any other aspect or combination of aspects listed herein the infusion pump apparatus includes the processor configured to determine a partial dose volume equal to the difference in volume between the programmed dose and the volume actually dispensed from the fluid supply.

In another aspect of the present disclosure, which may be used in combination with any other aspect or combination of aspects listed herein the infusion pump apparatus includes the processor configured to, if the partial dose volume equals zero, repeat in response to a subsequent request from the patient if a programmed lockout period has elapsed.

In another aspect of the present disclosure, which may be used in combination with any other aspect or combination of aspects listed herein the infusion pump apparatus includes the processor configured to, if the partial dose volume equals a value greater than zero, raise an alarm that the fluid supply is depleted and record, in a history log, the difference in volume between the programmed dose the volume of fluid actually dispensed.

In another aspect of the present disclosure, which may be used in combination with any other aspect or combination of aspects listed herein the infusion pump apparatus includes the processor configured to in response to the raised alarm that the fluid supply is depleted, and after a clinician exchanged the depleted fluid supply with a subsequent fluid supply, dispense, from the subsequent fluid supply, the partial dose volume.

In another aspect of the present disclosure, which may be used in combination with any other aspect or combination of aspects listed herein the infusion pump apparatus includes the processor configured to prevent any subsequent dispensing of fluid from the subsequent fluid supply for the programed lock out duration.

In another aspect of the present disclosure, which may be used in combination with any other aspect or combination of aspects listed herein the infusion pump apparatus includes the processor configured to repeat in response to a subsequent request from the patient if the programmed lockout period has elapsed.

In another aspect of the present disclosure, which may be used in combination with any other aspect or combination of aspects listed herein the infusion pump apparatus includes wherein the fluid supply and the subsequent fluid supply comprise the same fluid.

In another aspect of the present disclosure, which may be used in combination with any other aspect or combination of aspects listed herein the infusion pump apparatus includes wherein the fluid supply and the subsequent fluid supply comprise a syringe.

In another aspect of the present disclosure, which may be used in combination with any other aspect or combination of aspects listed herein the infusion pump apparatus includes wherein the fluid supply and the subsequent fluid supply contain an analgesic.

In another aspect of the present disclosure, which may be used in combination with any other aspect or combination of aspects listed herein the infusion pump apparatus includes wherein the patient request device is a hand held pendant with a button in operable communication with the processor of the infusion pump apparatus.

Additional features and advantages of the disclosed method and apparatus are described in, and will be apparent from, the following Detailed Description and the Drawings. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

The description will be more fully understood with reference to the following figures, which are presented as exemplary aspects of the disclosure and should not be construed as a complete recitation of the scope of the disclosure, wherein:

FIGS. 1A and 1B illustrate an example PCA pumping device, compatible with the presently disclosed method according to an aspect of the present disclosure.

FIG. 2 illustrates a block diagram of an electronic device, according to an example aspect of the present disclosure.

FIG. 3 illustrates a readout of the graphical user interface (GUI) of the PCA pump according to an example aspect of the present disclosure.

FIG. 4 illustrates a conceptual diagram of the timing of partial dose delivery according to an example aspect of the present disclosure.

### DETAILED DESCRIPTION

Turning now to the drawings, techniques are disclosed for new and innovative systems and methods for the delivery of partial medication in devices, such as medical devices. A variety of devices, such as infusion pumps, can be used to deliver treatments to patients. These treatments typically include delivering medication at a particular dose. Medical pumps may facilitate administration of intravenous therapy to patients both in and outside of a clinical setting. One type of medical pumps inside the clinical setting is a Patient-Controlled Analgesia (PCA) pump. PCA pumps typically deliver pain medication and allow patients to request medication delivery from the PCA pump, referred to as a PCA bolus, via a bolus cord attached to the pump. The PCA pump may also deliver pre-programmed intermittent boluses by programming the PCA pump to deliver a specific bolus volume with a specific time interval between two boluses. However, when a patient requests a PCA bolus and the volume remaining in the syringe is less than the requested bolus volume, the pump does not deliver any drug since the requested volume is not available.

Existing control techniques in PCA pumps emphasize accurate control of the dose volume delivered, however, in doing so, overlook a key issue and create several drawbacks. For example, when the volume in syringe of the PCA pump is less than the requested dose volume, the PCA pump does not deliver any bolus since the requested dose volume is not available in the syringe. As a result, the patient will not receive any dose until a clinician arrives at the patient's bedside and replaces the syringe or the user has to deliver pain medication via an alternate route. Existing control techniques also lead to the wastage of the remaining volume of medication in the syringe. Hospitals follow strict protocols to track the volume of medication being used or discarded to prevent unauthorized access to expensive and/or medication prone to abuse such as opioids. Systems and methods in accordance with aspects of the disclosure are capable of controlling a PCA or similar infusion pump to accurately deliver a programed dose volume in two partial bolus deliveries. These techniques can reduce the amount of time the patient is without the entirety of the programed dose, limiting pain and discomfort to the patient and ensure valuable medications are not wasted or put in a position where the medications could be misappropriated and abused.

A variety of systems and processes in accordance with aspects of the disclosure are described in more detail below.

Systems and Devices

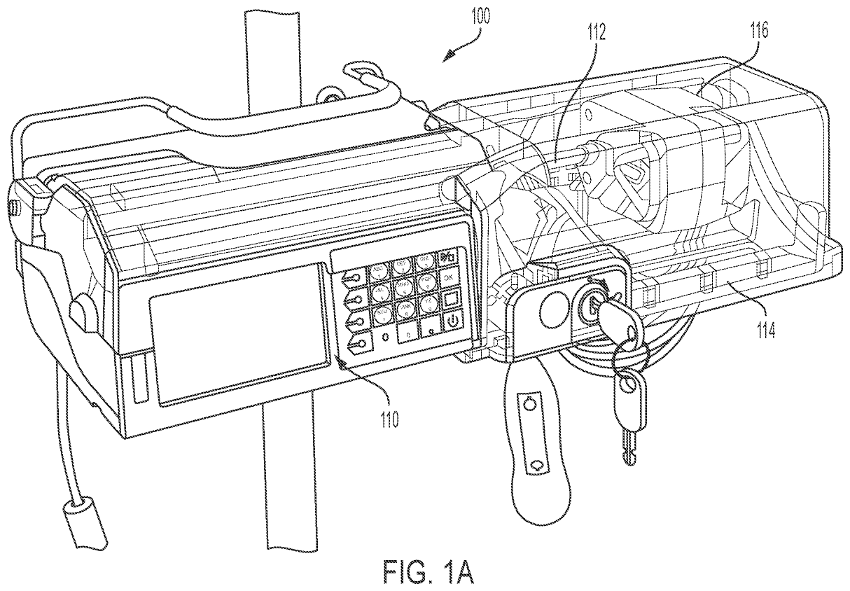

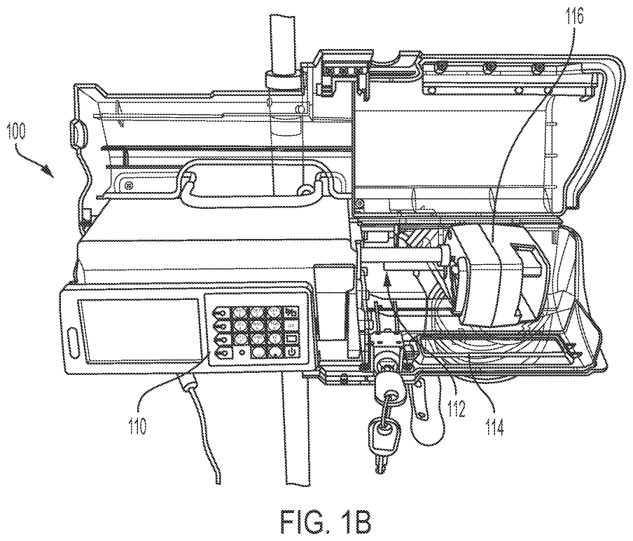

FIGS. 1A and 1B illustrate an example PCA pumping device, according to an aspect of the present disclosure. Device 100 (e.g. infusion pump) includes a graphical user interface 110 used to program a controller of the PCA pumping device 100. The device 100 can include any other electronic devices as appropriate. The device 100 includes a fluid supply 112, typically a syringe that is in fluid and operable connection with the device 100. The device 100 can be programmed to receive fluid from the fluid supply 112 and dispense the fluid at a specified rate utilizing a variety of processes as described herein.

The device 100 includes a case 114 that provides access to the fluid supply 112. The case 114 may be locked to prevent a patient (or anyone else other than an authorized clinician) from accessing the fluid supply 112. The case 114 may be transparent or opaque.

An actuator 116 is configured to cause fluid to move out of the fluid supply 112. For a syringe fluid supply 112, the actuator 116 is configured to apply force against a plunger (or a plunger flange) to cause the fluid to be dispensed to a patient via a patient IV line. The actuator 116 is controlled by a controller of the device 100, as discussed below.

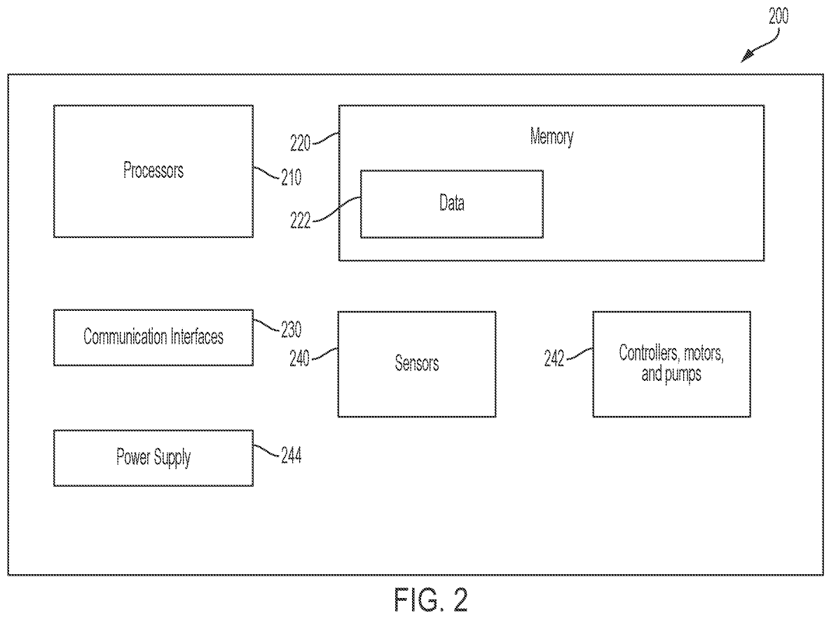

FIG. 2 illustrates a block diagram of an electronic device according to an example aspect of the present disclosure. The device 200 can include processors 210, memory 220, communication interfaces 230, sensors 240, controllers, motors, and pumps 242, and/or power supply 244. The processor 210 may also be referred to as a central processing unit (CPU). The processor 210 can include one or more devices capable of executing instructions encoding arithmetic, logical, and/or I/O operations. In many aspects, the processor 210 may be a single core processor that is typically capable of executing one instruction at a time (or process a single pipeline of instructions) and/or a multi-core processor that may simultaneously execute multiple instructions. In a variety of aspects, the processor 210 may be implemented as a single integrated circuit, two or more integrated circuits, and/or may be a component of a multi-chip module in which individual microprocessor dies are included in a single integrated circuit package and hence share a single socket.

The memory 220 can include any combination of volatile and/or non-volatile memory devices, such as RAM, ROM, EEPROM, or any other device capable of storing data. In a number of embodiments, the memory 220 stores a variety of data 222. In a variety of embodiments, the data 222 causes the device 200 to perform any of a variety of processes as described herein.

Communication interfaces 230 can include a network device (e.g., a network adapter or any other component that connects a computer to a computer network), a peripheral component interconnect (PCI) device, storage devices, disk drives, sound or video adaptors, photo/video cameras, printer devices, keyboards, displays, etc. The communications interfaces 230 can communicate via a variety of networks as appropriate. These networks can include a LAN (local area network), a WAN (wide area network), telephone network (e.g. Public Switched Telephone Network (PSTN)), Session Initiation Protocol (SIP) network, wireless network, point-to-point network, star network, token ring network, hub network, wireless networks (including protocols such as EDGE, 3G, 4G LTE, Wi-Fi, 5G, WiMAX, and the like), the Internet, and the like. A variety of authorization and authentication techniques, such as username/password, Open Authorization (OAuth), Kerberos, SecureID, digital certificates, and more, may be used to secure the communications.

Sensor devices 240 can include a variety of sensors to sense a variety of environmental and/or physical conditions. In several embodiments, the sensor devices 240 can be used to measure and/or record data regarding a patient being treated for a particular condition. In a variety of embodiments, sensor devices 240 can measure a motor position, a pump position, a voltage, a battery level, a fluid flow, and/or any other data as described herein. Controllers, pumps, and motors 242 can include any devices used to perform actions, such as electronic components, microcontrollers (such as PID controllers) motors, pumps, actuators, and the like. These actions can include, but are not limited to, adjusting an electrical output of a device, pumping fluid provided by a fluid supply, regulating the delivery of medicine (particularly within a desired flow rate accuracy), and the like. In a variety of aspects, the motor and pump are separate components. In many aspects, the motor and pump are a single component. In a number of aspects, some or all of the controllers, motors, and pumps 242 are implemented using processors 210. Power supply 244 can provide power to any of the components of device 200. The power supply 244 can include batteries, capacitors, transformers, charging circuitry, and/or any other device capable of providing AC and/or DC power to the components of device 200. In a variety of embodiments, the power supply 244 includes an AC/DC converter that converts AC power into 3.3V, 5V, and/or 12V DC power to the components of device 200. Charging circuitry of the power supply 244 can include any suitable charger, such as an AC charger, DC charger, solar panels, energy harvesters, and the like.

Although specific architectures for electronic devices in accordance with embodiments of the disclosure are conceptually illustrated in FIG. 2, any of a variety of architectures, including those that store data or applications on disk or some other form of storage and are loaded into memory at runtime, can also be utilized. Additionally, any of the data utilized in the system can be cached and transmitted once a network connection (such as a wireless network connection via the communications interface) becomes available. In a variety of embodiments, a memory includes circuitry such as, but not limited to, memory cells constructed using transistors, that store instructions. Similarly, a processor can include logic gates formed from transistors (or any other device) that dynamically perform actions based on the instructions stored in the memory. In several embodiments, the instructions are embodied in a configuration of logic gates within the processor to implement and/or perform actions described by the instructions. In this way, the systems and methods described herein can be performed utilizing both general-purpose computing hardware and by single-purpose devices.

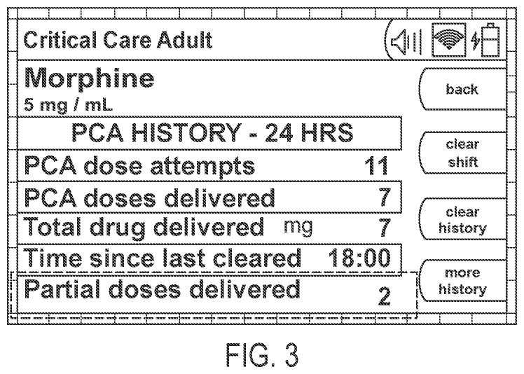

FIG. 3 illustrates a readout of the graphical user interface of the PCA pump according to an example aspect of the present disclosure. As discussed below in more detail below, an object of the present disclosure is to control the operation of pumps and manage the delivery of partial medication doses. In the process of delivering partial medication doses, the method and system disclosed herein detect and record the number of partial doses delivered. The graphical user interface (GUI) 300 of the disclosed pump provides the clinician or any other user of the pump an indication of how many partial doses have been delivered. Additionally, the GUI 300 may provide the clinician with additional information, for example, medication currently in fluid supply of the pump, the concentration of the medication, the number of doses attempted, the number of doses delivered, the total amount of medication delivered to the patient, the duration of time that has elapses since the pump was last cleared. The GUI 300 may also provide an indication of the current battery status of the battery powering the pump, the current network connectivity status of the pump, the current volume of alarm the pump is set at, and the time of day.

In an additional embodiment, the infusion pump apparatus includes a memory storing the programmed dose volume and capable of recording a history log. The memory may be any conventional computer readable medium or machine-readable medium, including volatile or non-volatile memory, such as RAM, ROM, flash memory, magnetic or optical disks, optical memory, or other storage media.

In an additional embodiment, the infusion pump apparatus includes a patient request device. For example, the patient request device may be a PCA bolus cord in operative communication with the pump, specifically a processor in communication with the pumping mechanism. Additionally, the patient request device may be a hand held pendant with a button in operable communication with the processor of the infusion pump apparatus. The patient request device may also be any handheld, corded or wireless devices capable of providing an input and/or instructions to the processor of the infusion pump apparatus.

In an additional embodiment, the infusion pump apparatus includes a processor in communication with the pumping mechanism, the memory and the patient request device. For example, the processor may be a single core processor that is typically capable of executing one instruction at a time (or process a single pipeline of instructions) and/or a multi-core processor that may simultaneously execute multiple instructions. In a variety of aspects, the processor may be implemented as a single integrated circuit, two or more integrated circuits, and/or may be a component of a multi-chip module in which individual microprocessor dies are included in a single integrated circuit package and hence share a single socket.

Additionally, the processor of the infusion pump apparatus is capable of performing several tasks including, dispensing the programmed dose volume from the fluid supply when the patient has made a request, via the patient request device. The processor of the infusion pump apparatus is also capable of determining a partial dose volume which is equal to the difference in volume between the programmed dose and the volume actually dispensed from the fluid supply in response to the patient's request. If the partial dose volume equals zero, the processor will instruct the pump to repeat the previous dispensing step in response to a subsequent request from the patient if a programmed lockout period has elapsed. Alternatively, if the partial dose volume equals a value greater than zero, the processor will instruct the pump to raise an alarm that the fluid supply is depleted and record, in a history log, the difference in volume between the programmed dose and the volume of fluid actually dispensed.

Next, in an additional embodiment, the processor of the infusion pump apparatus is capable of dispensing, from the subsequent fluid supply, the partial dose volume, only after the depleted fluid supply is exchanged with a subsequent fluid supply, in response to the raised alarm that the fluid supply is depleted. For example, a clinician can manually exchange the depleted fluid supply with a subsequent fluid supply. Also, in an additional embodiment, the infusion pump apparatus is capable of exchanging the depleted fluid supply with a subsequent fluid supply via automated means, not requiring human intervention. For example, the infusion pump may be configured to hold two fluid supplies and/or replace an active (depleted) fluid supply with another (fresh) fluid supply from a reserve area. In an embodiment, the fluid supply and the subsequent fluid supply may be the same fluid or different fluids. Additionally, the fluid supply and the subsequent fluid supply may be a syringe or any other container capable of containing fluid and engaging with the pumping mechanism.

Next, in an additional embodiment, the processor of the infusion pump apparatus is capable of preventing any subsequent dispensing of fluid from the subsequent fluid supply for the entirety of the programed lock out duration. As discussed above, unintended overdose as a result of the patient receiving excessive medication delivers can be extremely dangerous. Therefore, a programed lock out duration where the pump cannot delivered a subsequent dose is an aspect of the infusion pump apparatus that enhances patient safety.

Lastly, in an additional embodiment, the processor of the infusion pump apparatus is capable of repeat the previously disclosed processes in response to a subsequent request from the patient if the programmed lockout period has elapsed. For example, after the lockout period has elapsed, upon the patient making a request, via the patient request device, the processor will instruct the infusion pump apparatus to dispense the programmed dose volume from the fluid supply and repeat the subsequent processes associated with delivering partial boluses as disclosed herein.

Infusion Pump Apparatus

In an embodiment, an infusion pump apparatus for performing partial bolus deliveries includes a pumping mechanism configured to dispense a programmed dose volume from a fluid supply. For example, the pumping mechanism may include the components included in commercially available PCA pumps or any other infusion pump, such as the Baxter NovumIQ. These components may include but are not limited to means for retaining the fluid supply (syringe) within the pumping apparatus, a drive mechanism for depressing the syringe and driving fluid of the syringe, means for fluidly connecting the fluid supply to the pump, a plurality of sensors to monitor fluid supply levels and other conditions of the pumping apparatus. In an embodiment, the fluid supply may contain an analgesic or any other fluid or medication capable of being administered via an infusion pump.

In an additional embodiment, the infusion pump apparatus may include a memory storing the programmed dose volume and capable of recording a history log. The memory may be any conventional computer readable medium or machine-readable medium, including volatile or non-volatile memory, such as RAM, ROM, flash memory, magnetic or optical disks, optical memory, or other storage media. Additionally, in an embodiment, the memory is retrievable by a processor.

In an additional embodiment, the infusion pump apparatus may include a patient request device. For example, the patient request device may be a PCA bolus cord in operative communication with the pump, specifically a processor in communication with the pumping mechanism. Additionally, the patient request device may be a hand held pendant with a button in operable communication with the processor of the infusion pump apparatus. The patient request device may also be any handheld, corded or wireless devices capable of providing an input and/or instructs to the processor of the infusion pump apparatus.

In an additional embodiment, the infusion pump apparatus may include a processor in communication with the pumping mechanism, the memory and the patient request device. For example, the processor may be a single core processor that is typically capable of executing one instruction at a time (or process a single pipeline of instructions) and/or a multi-core processor that may simultaneously execute multiple instructions. In a variety of aspects, the processor may be implemented as a single integrated circuit, two or more integrated circuits, and/or may be a component of a multi-chip module in which individual microprocessor dies are included in a single integrated circuit package and hence share a single socket.

Additionally, the processor of the infusion pump apparatus may be capable of performing several tasks including, dispensing the programmed dose volume from the fluid supply when the patient has made a request, via the patient request device. The processor of the infusion pump apparatus may also be capable of determining a partial dose volume which is equal to the difference in volume between the programmed dose and the volume actually dispensed from the fluid supply in response to the patient's request. If the partial dose volume equals zero, the processor will instruct the pump to repeat the previous dispensing step in response to a subsequent request from the patient if a programmed lockout period has elapsed. Alternatively, if the partial dose volume equals a value greater than zero, the processor will instruct the pump to raise an alarm that the fluid supply is depleted and record, in a history log, the difference in volume between the programmed dose and the volume of fluid actually dispensed.

Next, in an additional embodiment, the processor of the infusion pump apparatus may be capable of dispensing, from the subsequent fluid supply, the partial dose volume, only after the depleted fluid supply is exchanged with a subsequent fluid supply, in response to the raised alarm that the fluid supply is depleted. For example, a clinician may manually exchange the depleted fluid supply with a subsequent fluid supply. Also, in an additional embodiment, the infusion pump apparatus may be capable of exchanging the depleted fluid supply with a subsequent fluid supply via automated means, not requiring human intervention. In an embodiment, the fluid supply and the subsequent fluid supply may be the same fluid or different fluids. Additionally, the fluid supply and the subsequent fluid supply may be a syringe or any other container capable of containing fluid and engaging with the pumping mechanism.

Next, in an additional embodiment, the processor of the infusion pump apparatus may be capable of preventing any subsequent dispensing of fluid from the subsequent fluid supply for the entirety of the programed lock out duration. As discussed elsewhere herein, unintended overdose resulting from the patient receiving excessive medication deliveries can be extremely dangerous. Therefore, a programed lock out duration where the pump cannot deliver a subsequent dose enhances patient safety.

Lastly, in an additional embodiment, the processor of the infusion pump apparatus may be capable of repeating the previously disclosed processes in response to a subsequent request from the patient if the programmed lockout period has elapsed. For example, after the lockout period has elapsed, upon the patient making a request, via the patient request device, the processor will instruct the infusion pump apparatus to dispense the programmed dose volume from the fluid supply and repeat the subsequent processes associated with delivering partial boluses as disclosed herein.

Device Operation Processes

As described herein, it is desirable for PCA pumps (and a variety of infusion pumps) to accurately dispense fluid a particular dose volume in two partial bolus deliveries. In a variety of aspects, an infusion pump is programmed to deliver a programed dose volume in either one complete bolus or two partial boluses from a fluid supply based on the available dose volume in the fluid supply. The infusion pump can utilize a controller, such as a PID controller, to accurately determine the operation of the motor and/or pump and dynamically correct for errors in the operation of the pump in performing the disclosed method.

In an embodiment, a computer-implemented method for delivering a programed dose volume in two partial boluses may include storing, in a memory of a pump, a programmed dose volume of a fluid from a fluid supply to be administered in response to a request from an operator, such as a patient. The fluid supply may be housed in a syringe that engages with the pump. In an additional example, the fluid supply may be housed in any other container that is capable of engaging with the pump. Additionally, the method includes instructing the pump when the request to dispense the programmed dose volume is received, via the controller, to perform a series of steps. In an example, the request to dispense the programmed dose volume is initiated by the patient via a bolus cord attached to the pump and is received via the pump controller.

First, in an embodiment, the method may include instructing the pump to begin dispensing the programmed dose volume of the fluid from the fluid supply. For example, if the fluid supply contains an analgesic and the programed dose volume is programed to 10 ml, the pump will begin dispensing the 10 ml of analgesic to the patient via an infusion line fluidly connecting the pump to the patient. The programed dose volume may vary based several factors, including but not limited to, the patient and the medication being delivered.

Next, in an embodiment, the method may include instructing the pump to receive an indication that the pump has stopped pumping. If the pumping has stopped, the pump will complete a series of steps including determining a partial dose volume that is equal to a difference in volume between the programmed dose and a volume of the fluid actually dispensed from the fluid supply. If the partial dose volume equals zero, the pump repeats in response to a subsequent request from the patient when a programmed lockout period has elapsed. In this example, the patient has received the entire programed dose volume in one bolus and, therefore, a second partial bolus is not necessary. In this example, the pump will prevent any subsequent dispensing of fluid from the fluid supply for a programed lock out duration. A lock duration is a period of time, during which no further patient requested bolus can be administered to the patient after a patient requested bolus has already been delivered to the patient. When the programed lock out duration has expired, the method may include repeating the previously disclosed processes in response to a subsequent request from the patient.

Next, in an embodiment, when the partial dose volume equals a value greater than zero, the method may include instructing the pump to generate an alarm indicating that the fluid supply is depleted and record, in a history log stored in the memory, the difference in volume between the programmed dose and the volume of the fluid dispensed from the fluid supply. In this example, the patient has not received the entire programed dose volume in one bolus and, therefore, a second partial bolus is necessary. Additionally, in this embodiment, the method may include providing a prompt to exchange the depleted fluid supply with a subsequent fluid supply, in response to the alarm that the fluid supply is depleted. The prompt may be provided via a graphical user interface (GUI) on the pump that instructs a clinician (visually or audibly) to manually exchange the depleted fluid supply with a subsequent fluid supply. In an embodiment, the fluid supply and the subsequent fluid supply comprise the same fluid and the subsequent fluid supply may also be housed in a syringe configured to engage with the pump. In an example, the method includes the pump exchanging the depleted fluid supply with a subsequent fluid supply via automated means, not requiring human intervention.

Next, in an embodiment, the method may include instructing the pump to dispense, from the subsequent fluid supply, the partial dose volume after receiving an indication that the subsequent fluid supply is fluidly coupled to the pump. For example, according to previous steps of the disclose method, if the programed dose volume equals 10 ml of fluid and the dose volume actually delivered in the first bolus equals 7 ml of fluid, the partial dose volume equals 3 ml of fluid. Therefore, after receiving an indication that the subsequent fluid supply is fluidly coupled to the pump, the pump will dispense, from the subsequent fluid supply, 3 ml of fluid.

Lastly, in an embodiment, the method may include instructing the pump to prevent any subsequent dispensing of fluid from the subsequent fluid supply for the duration of the programed lock out period. This prevents inadvertent overdoses and excessive treatment that may be harmful to the patient. Additionally, the controller of the pump instructs the pump to repeat the method in response to a subsequent request from the patient if the programmed lockout period has elapsed. For example, after the lockout period has elapsed, the patient may request a programed dose volume, via the bolus cord in communication with the pump, to initiate a subsequent dose of medication.

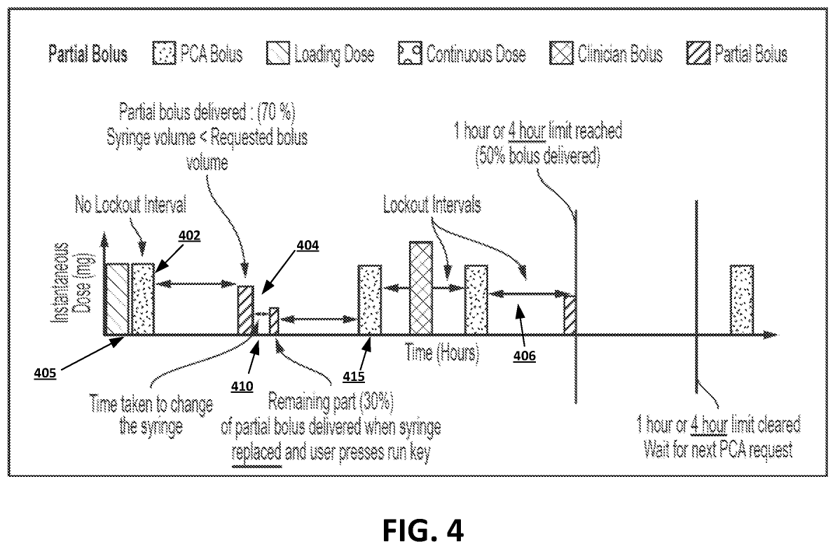

FIG. 4 illustrates a flowchart of a partial bolus delivery process according to an example aspect of the present disclosure. Although the process 400 is described with reference to the flowchart illustrated in FIG. 4, it will be appreciated that many other methods of performing the acts associated with the process 400 may be used. For example, the order of some of the blocks may be changed, certain blocks may be combined with other blocks, one or more blocks may be repeated, and some of the blocks described are optional. The process 400 may be performed by processing logic that may include hardware (circuitry, dedicated logic, etc.), software, or a combination of both. In a variety of aspects, pulse mode operation processes can be performed by a controller operating a motor and/or pump within an infusion pump device.

Also illustrated in FIG. 4, the disclosed methods and systems allow for the delivery of pre-programmed intermittent boluses by programming the controller to deliver specific bolus volumes and time intervals between two boluses. The bolus volume and minimum time interval between the boluses are pre-programmed by the clinician. The pre-programmed bolus may be independent of patient requested bolus.

FIG. 4 also depicts the boluses delivered to a patient over time. For example, at 405, a PCA Bolus dosage 402 is loaded and delivered to a patient. Later, a partial bolus 404 is delivered to the patient at 410. The amount of the requested bolus may exceed the syringe volume, at which point the syringe may be replaced and the remainder of the requested amount may be delivered to a patient. At 415 and at a later time, a bolus may be delivered to the patient at request of the patient. There may be lockout intervals, e.g. 406, during which the system will not deliver a bolus despite receiving a request from the patient.

Various scenarios contemplated by the method and system disclosed herein are provided in Table 1:

TABLE 1

| | | | PCA + | | |
|---|---|---|---|---|---|
| | Description | PCA Only | Continuous | Pros | Cons |
| Problem: Don't | Pump does not | No bolus | No bolus | Easier to keep | Patient will be in |
| allow bolus | respond. | | | track of drug | pain Drug is |
| | | | | delievered | wasted |
| Proposed | Programmed bolus | 10 ml | 10 ml | Patient will get | Difficult to track |
| Solution 1: | delivered in 2 parts. | delivered | delivered | some drug | partial bolus |
| Deliver partial | Log event in History | in 4 mins | in less than | | since user is |
| bolus | log | | 4 mins | | unaware of it |
| Proposed | Control system raises | 10 ml | 10 ml | User is alerted | Difficult to track |
| Solution 2: Raise | an alarm and delivers | delivered | delivered | by alarm and is | number of |
| an alarm #1 | partial bolus. Log | in 4 mins | in less than | aware of partial | partial boluses |
| | event in History log | | 4 mins | bolus | |

In the problem scenario noted in Table 1 above, the fluid pump will not deliver the PCA bolus requested by the patient because of a lockout interval. In this scenario, the fluid pump determines that a patient has received the maximum number of boluses during a time period and that no further boluses are permitted during this lockout interval. As a result, the patient will have to wait until the lockout interval expires. In this case, the remaining drug in the semi-depleted syringe is not utilized and wasted if it is replaced with a new syringe. Opioids, for example, are critical and expensive drugs and every small amount should be utilized.

To overcome this problem scenario, proposed solution one allows the pump to deliver remaining amount in the syringe after which a syringe empty alarm is triggered. This alerts the clinician to replace the syringe with a full syringe and continue the therapy delivery. The patient receives a partial amount of the drug, however, there is minimum interruption in therapy. Additionally, the entire volume in the syringe is delivered, resulting in no wastage of the drug. Once the syringe is replaced, the control system automatically delivers the pending bolus volume equal to the difference in volume between the programmed dose and the volume of the fluid dispensed from the fluid supply. Thus, the patient receives the entire bolus volume in two partial deliveries helping in maintaining the analgesic effect. However, the user may not be aware of the number of partial boluses. To remedy this issue, the number of partial boluses is recorded in a history log as shown in a GUI screen so that the user knows the exact number of partial boluses in comparison to whole boluses delivered. Even though the entire bolus volume is delivered in two partial parts, it will be counted as two partial boluses in the history log.

Proposal two is an update to proposal one, wherein the control system will raise an alarm as soon as the patient requests a bolus and the volume in syringe is insufficient to deliver the requested (i.e. programed) amount of medication. This alerts the clinician as well as logs the same in the history log and displays via the pumps GUI, ensuring the clinician is aware of the partial bolus during its delivery as well as option to review all partial boluses in the history log.

It will be appreciated that all of the disclosed methods and procedures described herein can be implemented using one or more computer programs, components, and/or program modules. These components may be provided as a series of computer instructions on any conventional computer readable medium or machine-readable medium, including volatile or non-volatile memory, such as RAM, ROM, flash memory, magnetic or optical disks, optical memory, or other storage media. The instructions may be provided as software or firmware and/or may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures. As will be appreciated by one of skill in the art, the functionality of the program modules may be combined or distributed as desired in various aspects of the disclosure.

Although the present disclosure has been described in certain specific aspects, many additional modifications and variations would be apparent to those skilled in the art. In particular, any of the various processes described above can be performed in alternative sequences and/or in parallel (on the same or on different computing devices) in order to achieve similar results in a manner that is more appropriate to the requirements of a specific application. It is therefore to be understood that the present disclosure can be practiced otherwise than specifically described without departing from the scope and spirit of the present disclosure. Thus, embodiments of the present disclosure should be considered in all respects as illustrative and not restrictive. It will be evident to the annotator skilled in the art to freely combine several or all of the embodiments discussed here as deemed suitable for a specific application of the disclosure. Throughout this disclosure, terms like “advantageous”, “exemplary” or “preferred” indicate elements or dimensions which are particularly suitable (but not essential) to the disclosure or an embodiment thereof, and may be modified wherever deemed suitable by the skilled annotator, except where expressly required. Accordingly, the scope of the invention should be determined not by the embodiments illustrated, but by the appended claims and their equivalents.

What is claimed is:

1. A computer-implemented method, comprising:
storing, in a memory of a pump, a programmed dose volume of a fluid from a fluid supply to be administered in response to a request from an operator; and
when the request to dispense the programmed dose volume is received in a controller of the pump, instructing, via the controller, the pump to:
begin dispensing the programmed dose volume of the fluid from the fluid supply,
receive an indication that the pump has stopped pumping,
determine a partial dose volume equal to a difference in volume between the programmed dose and a volume of the fluid actually dispensed from the fluid supply,
when the partial dose volume equals zero, repeat in response to a subsequent request from the patient when a programmed lockout period has elapsed,
when the partial dose volume equals a value greater than zero, generate an alarm that the fluid supply is depleted and record, in a history log stored in the memory, the difference in volume between the programmed dose and the volume of the fluid dispensed from the fluid supply,
in response to the alarm that the fluid supply is depleted, provide a prompt to exchange the depleted fluid supply with a subsequent fluid supply,
after receiving an indication that the subsequent fluid supply is fluidly coupled to the pump, dispense, from the subsequent fluid supply, the partial dose volume, and
prevent any subsequent dispensing of fluid from the subsequent fluid supply for the programed lockout period.

2. The computer-implemented method of claim 1, wherein the fluid supply and the subsequent fluid supply comprise the same fluid.

3. The computer-implemented method of claim 2, wherein the fluid supply and the subsequent fluid supply are housed in a syringe configured to engage with the pump.

4. The computer-implemented method of claim 2, wherein the fluid supply and the subsequent fluid supply contain an analgesic.

5. The computer-implemented method of claim 1, wherein the controller of the pump is configured to instruct the pump to repeat the method in response to a subsequent request from the patient when the programmed lockout period has elapsed.

6. The computer-implemented method of claim 1, further comprising displaying information related to the dispensing of fluid over a time period.

7. The computer-implemented method of claim 6, wherein the displayed information includes information from the history log.

8. The computer-implemented method of claim 1, further comprising, in response to the alarm indicative that the fluid supply is depleted, automatically exchanging the fluid supply for another fluid supply comprising the same fluid.

9. An infusion pump apparatus comprising:
a pumping mechanism configured to dispense a programmed dose volume from a fluid supply;
a memory storing the programmed dose volume and configured to record a history log;
a patient request device; and
a processor in communication with the pumping mechanism, the memory, and the patient request device, the processor configured to:
when the patient has made a request, via the patient request device, dispense the programmed dose volume from the fluid supply,
determine a partial dose volume equal to the difference in volume between the programmed dose and the volume actually dispensed from the fluid supply,
when the partial dose volume equals zero, repeat in response to a subsequent request from the patient if a programmed lockout period has elapsed,
when the partial dose volume equals a value greater than zero, raise an alarm that the fluid supply is depleted and record, in a history log, the difference in volume between the programmed dose the volume of fluid actually dispensed,
in response to the raised alarm that the fluid supply is depleted, and after a clinician exchanged the depleted fluid supply with a subsequent fluid supply, dispense, from the subsequent fluid supply, the partial dose volume,
prevent any subsequent dispensing of fluid from the subsequent fluid supply for the programed lockout period, and
repeat in response to a subsequent request from the patient when the programmed lockout period has elapsed.

10. The infusion pump apparatus of claim 9, wherein the fluid supply and the subsequent fluid supply comprise the same fluid.

11. The infusion pump apparatus of claim 10, wherein the fluid supply and the subsequent fluid supply comprise a syringe.

12. The infusion pump apparatus of claim 10, wherein the fluid supply and the subsequent fluid supply contain an analgesic.

13. The infusion pump apparatus of claim 9, wherein the patient request device is a hand held pendant with a button in operable communication with the processor of the infusion pump apparatus.

14. The infusion pump apparatus of claim 9, wherein the patient request device is a bolus cord in operable communication with the pumping mechanism.

15. The infusion pump apparatus of claim 9, further comprising a display interface that displays information related to the dispensing of fluid over a predetermined time period.

16. The infusion pump apparatus of claim 15, wherein the displayed information includes information related to at least one of the fluid supply and the subsequent fluid supply.

17. The infusion pump apparatus of claim 9, wherein, in response to the raised alarm that the fluid supply is depleted, the processor is further configured to cause the pump to automatically exchange the fluid supply for the subsequent fluid supply.

18. The infusion pump apparatus of claim 9, wherein the partial dose volume is determined based on at least one input from one or more sensors configured to detect physical and/or environmental conditions.

19. The infusion pump apparatus of claim 9, further comprising a case to provide access to the fluid supply.

20. The infusion pump apparatus of claim 19, wherein the case includes a lock to prevent patient access to the fluid supply.
